(19) United States
(12) Patent Application Publication
(10) Pub. No.: US 2009/0162870 A1
(43) Pub. Date: Jun. 25, 2009

(54) FATTY ACID SYNTHASE IN LIVER DISEASE

(75) Inventors: Susan M. Medghalchi, Ellicott, MD (US); Francis P. Kuhajda, Baltimore, MD (US)

Correspondence Address: Patentique PLLC, P.O. Box 5803, Bellevue, WA, 98006, US

(73) Assignee: FASgen Diagnostics, LLC, Baltimore, MD (US)

(21) Appl. No.: 12/038,803

(22) Filed: Feb. 27, 2008

Related U.S. Application Data
(60) Provisional application No. 60/891,928, filed on Feb. 27, 2007.

(51) Int. Cl. G01N 33/573

(52) U.S. Cl. 435/7.4

(57) ABSTRACT

Methods and compositions for detecting elevated fatty acid synthase (FAS) expression in the liver of a subject are disclosed. The detection may be of expression in liver cells per se or in a bodily fluid of a subject. Also disclosed are methods for identifying the presence or absence of liver disease or pathology in relation to elevated FAS. The disclosed methods may be practiced with various compositions comprising reagents for detecting FAS expression as described herein.

### RELATED APPLICATIONS

This application claims benefit of priority from U.S. Provisional Patent Application 60/891,928, filed Feb. 27, 2007, which is hereby incorporated in its entirety as if fully set forth.

### FIELD OF THE DISCLOSURE

Methods and compositions for detecting elevated fatty acid synthase (FAS) expression in the liver of a subject are disclosed. The detection may be of expression in liver cells per se or in a substance of a bodily fluid of a subject. Also disclosed are methods for identifying the presence or absence of liver disease or pathology in relation to elevated FAS. The disclosed methods may be practiced with various compositions comprising reagents for detecting FAS expression as described herein.

### BACKGROUND OF THE DISCLOSURE

Nonalcoholic steatohepatitis (NASH), also known as nonalchoholic fatty liver disease (NAFLD), is a chronic liver disease characterized by a spectrum of pathology from simple steatosis (fatty liver), to NASH/NAFLD, progressing to established cirrhosis (1). The diagnosis of NASH is established on liver biopsy where the pathology is essentially indistinguishable from alcoholic hepatitis. Steatosis, foci of acute and chronic inflammation with hepatocellular injury, Mallory's hyaline, and variable amounts of fibrosis are among the hallmarks of the disease (2) and must occur in the absence of significant alcohol consumption.

NASH has been subclassified into two groupings, secondary and primary (1). Secondary NASH occurs in the setting of fatty liver disease as the result of a proximate cause such as jejuno-ileal bypass surgery, drugs, hepatotoxins, or diseases such as lipodystrophy, Weber-Christian disease, or HIV. Primary NASH is associated with obesity, Type II diabetes, dyslipidemia and insulin resistance, part of the constellation of signs and symptoms of the metabolic syndrome, or may be idiopathic.

In its early stages NASH is essentially asymptomatic, therefore, it is important to establish the diagnosis before the onset symptoms which are commonly related to the onset of cirrhosis. It is also possible for cirrhosis to develop in NASH without the appearance of symptoms. Although the liver biopsy remains the gold-standard for the diagnosis of NASH, needle biopsies are fraught with both sampling error and potential misinterpretation (3, 4). Moreover, liver biopsy and imaging techniques such as magnetic resonance spectroscopy (MRS) are hindered by cost and impracticality as screening tests. Thus, there have been attempts to utilize the common and relatively inexpensive liver function tests performed on serum to detect NASH.

There are a number of assays used to detect the presence of liver enzymes in the serum which may be used alone or in combination as markers of liver disease. Presence of these enzymes in the blood indicates hepatocellular injury. These enzymes include: AST (aspartate aminotransferase), ALT (alanine aminotransferase), alkaline phosphatase (ALP), GGT (gamma-glutamyl transferase), and lactate dehydrogenase (LDH). While elevations of these enzymes in the blood indicate hepatocellular injury, the cannilcular location of ALP and GGT may indicate an obstructive process such as biliary obstruction from stones or tumors (5).

Unfortunately, levels of liver enzymes are both insensitive and nonspecific for chronic liver disease in general, and NASH specifically (1, 6-10). For example, in a recent study by Kunde et al, the sensitivity and specificity of serum ATL elevations to detect NASH in 233 women with class II/II obesity were 74% and 42% using ALT>19 U/L as the cutoff for normal. Changing the cutoff value to ALT>30 U/L increased the sensitivity and specificity to 42% and 80%. However, the authors concluded that the diagnostic utility of ALT to identify NASH remains poor (6). In a longitudinal study of 106 patients with NASH, Fassio et al showed that neither ALT nor AST/ALT ratio were useful to predict progression of NASH (8). In a study of NASH in 51 patients with normal ATL levels compared to 50 patients with elevated ALT levels, the entire spectrum of disease was seen in the patients with normal ALT levels ranging from steatosis to established cirrhosis. Thus, a normal ALT value does not preclude progression of NASH to advanced fibrosis (10). To examine this issue another way, 119 patients who were hepatitis B and C virus negative with elevated ALT levels found at the time of blood donation were studied for the cause of elevated ALT levels. Obesity (30.2%) and alcoholism (28.6%) were most frequently associated with the ALT elevation. Liver histology in 40 patients showed steatosis (35%) steatohepatitis (30%), non-specific hepatitis (12.5%) and normal liver in 15%; with one case each of cirrhosis, hemochromotosis, and portal fibrosis. Again, ALT elevations were not highly predictive of NASH in this population (11). In summary, there is no practical test useful to screen for the presence of NASH.

Fatty acid synthase (FAS) has been of interest as a drug target for human cancer treatment and a biomarker for cancer diagnosis. FAS is expressed in a high percentage of most common human tumors such as lung, prostate, colon, breast and ovary (12). Inhibition of FAS induces apoptosis in human cancer cells (13) and small molecule FAS inhibitors inhibit the growth of human cancer xenografts (14, 15). In addition, elevated levels of FAS in the blood of breast cancer patients has been reported (16-18). There are also reports of significantly elevated FAS levels in patients with lung, breast, ovary, pancreas, and colon cancer compared to controls (19). Recently, a newly configured FAS ELISA assay has confirmed these prior serological studies (FIG. 1).

Citation of the above documents is not intended as an admission that any of the foregoing is pertinent prior art. All statements as to the date or representation as to the contents of these documents is based on the information available to the applicant and does not constitute any admission as to the correctness of the dates or contents of these documents.

### SUMMARY OF THE DISCLOSURE

Disclosed herein are methods and compositions for detecting or identifying conditions of the liver, including liver disease and pathology comprising a variety of etiologies. The methods may be advantageously combined with, or followed by, methods to treat the identified liver condition, disease or pathology. Aspects of the methods, and activities of the compositions, include detecting or measuring fatty acid synthase (FAS) expression in the liver of a subject, such as a human patient. Embodiments of the disclosure include application of a detected or measured FAS expression level in the diagnosis of disease and the selection of treatment for the disease.

In one aspect, the disclosure includes a method of detecting or measuring fatty acid synthase (FAS) expression in the liver of a subject. The detecting or measuring may be of the level, or amount, of FAS expression. In some embodiments, the detecting or measuring is of FAS protein, or a fragment thereof. Non-limiting embodiments of the method comprise the use of an FAS containing sample of material from the subject. In some embodiments, the FAS containing sample comprises a bodily fluid, such as blood or serum. In other non-limiting embodiments, the FAS containing sample comprises one or more liver cells from the subject, a homogenate of liver cells, or a FAS containing extract of liver cells.

In some embodiments, the sample may be from a subject thought, believed, or expected to have a liver condition. Alternatively, the subject may be suspected of having such a condition. In other embodiments, the subject may be one that is suspected, thought, believed, or diagnosed as being cancer-free. As an additional alternative embodiment, the subject may be one who has already been diagnosed with a liver condition characterized by elevated FAS expression, and the methods of the disclosure are used to confirm or provide additional support for the diagnosis.

In further embodiments, a method of detecting or measuring FAS expression may be used to determine or identify an elevated level of FAS in the liver of a subject. Alternatively, the method may be used to determine or identify a non-elevated, or normal, level of FAS in a subject's liver. The level of FAS may be determined to be elevated or non-elevated in comparison to FAS levels in samples from subjects who are free of liver disease or pathology.

Some embodiments of the method are based upon detecting or measuring one or more components in the sample that reflects the FAS expression level therein. In some cases, the component may be FAS protein, or a fragment thereof. In such embodiments, the method may comprise the use of a reagent that binds the FAS protein or fragment thereof. In other embodiments, the component may be a cellular factor or intermediate reflective of FAS expression. In such embodiments, the method may comprise the use of one or more reagents to detect or measure the factor or intermediate. As a non-limiting example, the component may be FAS protein encoding mRNA, and the reagent may be a nucleic acid probe to detect the mRNA. Alternatively, a combination of reagents for reverse transcription polymerase chain reaction (RT-PCR), such as primer and probe molecules, may be used. An additional alternative is the use of quantitative PCR, such as to detect FAS cDNA after reverse transcription of mRNA.

In another aspect, the disclosure includes a method of diagnosing the presence or absence of a liver condition based upon a detected or measured level of FAS. In some embodiments, the diagnosis is of the presence of liver disease or pathology based on elevated FAS expression. In other embodiments, the diagnosis is of the absence of liver disease or pathology based on non-elevated FAS expression. Non-limiting examples of liver disease or pathology include steatohepatitis (or non-alcoholic steatohepatitis, NASH); alcoholic hepatitis; liver toxicity; viral infection of the liver, or viral hepatitis; autoimmune hepatitis; cryptogenic cirrhosis; hepatic necrosis following hypoperfusion; and hepatitis resulting from other disease, or secondary NASH.

In a further aspect, the disclosure includes a method of diagnosing fat accumulation in the liver of a subject based upon a detected or measured FAS expression level. In some embodiments, the diagnosis of the presence of fat accumulation in the liver of a subject is based upon elevated FAS expression. Alternatively, a diagnosis of the absence of fat accumulation in the liver of a subject may be based upon the detection or measurement of non-elevated level of FAS expression. In some embodiments, these methods may be used in combination the detection or measurement of liver inflammation. The absence of inflammation is indicative of the presence of steatosis, while the presence of inflammation, such as focal acute inflammation in the liver lobule with associated hepatocellular injury, is indicative of the presence of steatohepatitis. It has been reported that mild chronic inflammation of the portal triad may be present in patients with steatosis.

In an additional aspect, methods of the disclosure may be used in combination with other medical or clinical methods as part of a method of differential diagnosis. Such a method may comprise the practice of the FAS related assays as described herein in combination with other assays to advance a medical diagnostic process by including or excluding other possible disease conditions.

In a yet additional aspect, the disclosure includes a method of selecting or applying treatment or therapy based upon a diagnosis of a liver condition as described herein. In some embodiments, the treatment or therapy is one that is known to, or thought by, the skilled person for alleviating or improving a symptom or aspect of the diagnosed condition. Non-limiting examples include treatments and therapies recognized by a clinician or other medical practitioner and thought or known to be of benefit against the diagnosed disease or pathology.

In a yet further aspect, the disclosure includes a method of preparing a sample of biological material from a subject as described herein. The preparation may be by any means or methods known to the skilled person. In some embodiments, a sample of biological fluid may be prepared and then used in a method disclosed herein. In other embodiments, a sample containing liver cells, or material therefrom, may be prepared and used in a method described herein.

The disclosure further includes compositions for the practice of the methods disclosed herein. Non-limiting embodiments include reagents for the detection of FAS protein, or fragments thereof, or for the detection of FAS encoding nucleic acids. Of course compositions comprising one or more such reagents are also disclosed. Additional materials include complexes comprising a reagent for detecting FAS expression and a ligand bound by the reagent. The reagent and ligand form a “binding pair” of the disclosure such that a complex of the disclosure may comprise such a “binding pair.” Non-limiting examples of ligands include components and molecules in a biological fluid or a liver cell that are bound by the reagent. In some embodiments, the complexes are isolated or purified from one or more molecules normally found with the ligand.

The details of additional embodiments are set forth in the accompanying drawings and the description below. Other features, objects, and advantages of the embodiments will be apparent from the drawings and detailed description, and from the claims.

### DEFINITIONS

The term “liver condition” and variants thereof as used herein refer to conditions of the liver in a subject that are not those of a normal or disease-free individual. The condition may be that of an abnormal liver or that of an undesired physiological state. One non-limiting example is fatty liver. The term further includes conditions such as liver disease and liver pathology.

“Liver disease” and variants thereof as used herein refer to conditions of the liver in a subject that disrupts its normal level or characteristic function. In some cases, the disruption is an interruption, cessation, or disorder of liver function. In other cases, the disruption is an incorrect or aberrant level or character of liver function. The disease may be the result of a genetic factor or state in the subject or one or more etiological agents.

The term “liver pathology” and variants thereof as used herein refer to disease conditions of the liver which comprise a structural component that may be identified by examination of liver cells and/or tissue. Pathology is often a state or condition that may be identified by the study of tissue, organs, and/or bodily fluids. The study may be based on visual inspection, such as under a microscope, that is optionally augmented by staining and/or immunohistochemistry.

As used herein, “steatohepatitis” includes both non-alcoholic steatohepatitis (NASH) and alcoholic steatohepatitis.

The term “human fatty acid synthase” or “hFAS” or refers to the polypeptide previously identified as a cancer related antigen in U.S. Pat. No. 5,759,791 and the patent applications from which it depends. The antigen is also referred to as OA-519 in the field and is defined by the Nomenclature Committee of the International Union of Biochemistry and Molecular Biology (NC-IUBMB) as fatty acid synthase (E.C. 2.3.1.85), as described at www.chem.qmul.ac.uk/iubmb/enzyme/. The terms are not limited to a particular human fatty acid synthase by amino acid sequence but rather any hFAS or fragment thereof that is recognized by an antibody of the disclosure. The terms also refer to hFAS proteins or peptides, including fragments of a full length sequence, which remain intracellular as well as cell-free forms found in extracellular environments and bodily fluids. In some cases, a fragment of a full length hFAS is one which is indicative of (unique to) full length hFAS. The terms “FAS polypeptide”, “FAS peptide” and “FAS protein” as used herein refer to a polymer of amino acid residues that is all or part of FAS in its entirety. These terms also encompass polymers containing conservative amino acid substitutions such that the polymer in its entirety retains its functionality, such as the functionality of being recognized by an anti-FAS antibody of the disclosure.

The term “contacting” refers to placement in direct physical association, such as the placement of an antibody of the disclosure with a hFAS polypeptide such that formation of a complex of these two components may result.

The term “elevated FAS level”, or variations thereof, relates to a qualitative or quantitative assessment of the presence or absence of an FAS polypeptide, or a complex comprising an FAS polypeptide, in a sample or other material. The phrase may also be considered the detection of the presence of an FAS polypeptide above a specific level, such as, but not limited to, a level above background noise or the level in a reference cell or sample (including a cell or sample from a normal subject). The use of “determining” or “detecting” the level of an FAS polypeptide as used herein refers to the assessment of the amount of a polypeptide at a quantitative or semi-quantitative level. The assessment need not be absolutely accurate but may instead be approximate.

The terms “conjugate”, “bond”, “link”, and variations thereof refer to the physical attachment of two entities via formation of at least one covalent bond. In some situations, they refer to making two polypeptides into one contiguous polypeptide molecule. In the context of the disclosure, the terms include reference to joining an antibody moiety to a solid phase support or other solid phase material, including the surface of a solid phase material, as well as another molecule. The formation of a covalent bond may be by use of a chemical reaction to form the bond. The term “support” refers to conventional supports such as beads, particles, dipsticks, fibers, filters, membranes and silane or silicate supports such as glass slides. Conjugated antibodies of the disclosure include, but are not limited to, antibodies that are attached to a label.

### BRIEF DESCRIPTION OF THE DRAWINGS

FIG. 1 illustrates serum FAS levels in patients with breast, ovarian, prostate, lung and colon cancer compared to normal controls. Normal Male refers to results of 0.21+0.27 ng/ml (mean±standard deviation) where n=10. Normal Female refers to results of 0.45±0.67 ng/ml (mean±standard deviation) where n=20. “Ovary” refers to ovarian cancer cases where n=13. “Breast” refers to breast cancer cases where n=12. “Colon” refers to colon cancer cases where n=10. “Prostate” refers to prostate cancer cases where n=13. “Lung” refers to lung cancer cases where n=11. The upper normal limit for males and females is 1.02 and 2.46 ng/ml (mean+3 standard deviations), respectively.

FIG. 2 illustrates FAS expression in relation to steatosis and steatohepatitis in liver biopsies from 38 (n=38) obese subjects. Part A shows a plot of fatty liver score versus steatohepatitis score where p<0.00011-way ANOVA, and *** indicates p<0.001 by two-tailed t-test. Part B shows a plot of FAS expression score versus fatty liver score where p<0.011 1-way ANOVA, * indicates p<0.05, and ** indicates p<0.01 by two-tailed t-test. Part C shows a plot of FAS expression score versus steatohepatitis score where p<0.0003 1-way ANOVA, and ** indicates p<0.01 by two-tailed t-test.

FIG. 3 shows FAS expression in samples of normal liver, steatosis, steatohepatitis, and cirrhosis.

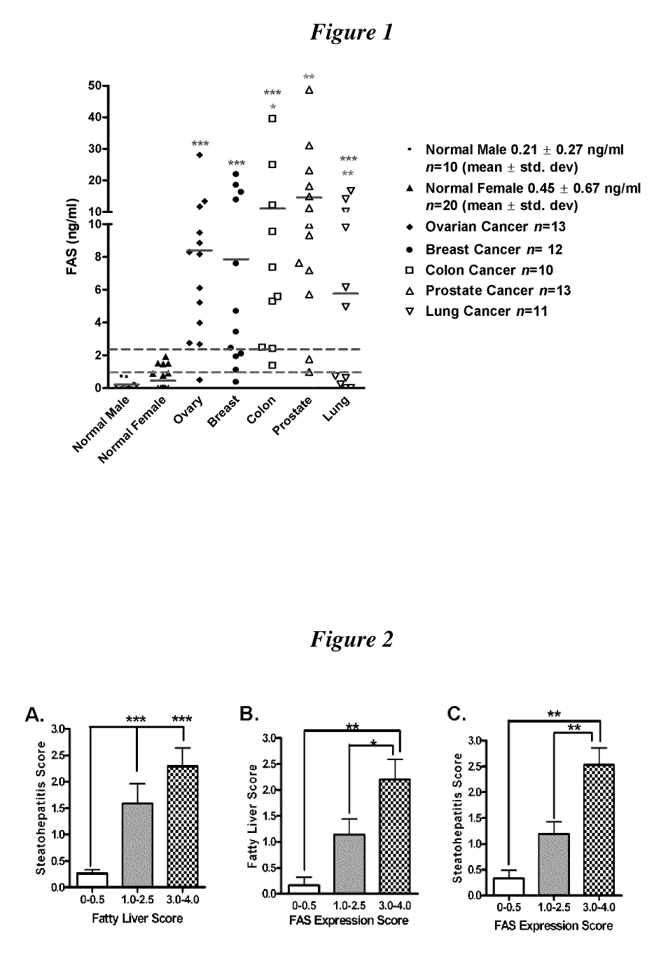

### DETAILED DESCRIPTION OF MODES OF PRACTICING THE DISCLOSURE

This disclosure includes methods and compositions for the detection of liver conditions in a subject. Detection of the conditions may be used to improve the diagnosis and/or treatment of disease in a subject. The methods may be conducted in a qualitative manner, such as by detection of FAS expression at or above a certain level. Alternatively, the methods may be conducted in a quantitative manner, such as by measurement of the level or amount of FAS expression.

The disclosure includes a method of detecting elevated FAS in the liver of a subject. In some embodiments, the method may comprise analysis of FAS expression in one or more liver cells. In other embodiments, the method may comprise analysis of FAS that has been released from the liver, such as FAS in a bodily fluid of a subject. In embodiments where the bodily fluid is serum, FAS levels are disclosed herein as not correlating with ALT, AST, ALP, total bilirubin, total protein, or albumin levels. In further embodiments, the serum has been stored for about 1, about 1 to 2, about 2, about 2-3, about 3, about 3-4, or about 4 days at 4° C.

Non-limiting examples of analysis include detecting or measuring FAS expression, or the FAS level, in the liver of a subject. The detecting or measuring may be made directly, such as by detection of FAS protein or a fragment thereof. Alternatively, the detecting or measuring may be made indirectly, such as via an intermediate indicative of FAS expression.

The detecting or measuring may be of a sample, such as a sample of one or more liver cells from a subject or a sample of bodily fluid from a subject. In some embodiments, the subject is human. In other embodiments, the subject is a non-human animal, such as those susceptible to metabolic liver syndrome (see Hansen, R. J. et al. “Avian fatty liver hemorrhagic syndrome: a comparative review.” Adv Vet Sci Comp Med 37: 451-468, 1993.) Non-limiting examples include birds (e.g. chickens, ducks, geese, and other agriculturally important avian species), mammals (e.g. cows, pigs, and other agriculturally important quadrapeds), and animals for human companionship (e.g. cats, dogs, etc.).

In some embodiments, the subject is a human patient, such as a non-obese person, who will generally have a serum FAS level that is lower than that of an obese person. Thus elevated serum FAS level in a non-obese person indicates the presence of steatosis or steatohepatitis. Moreover, an obese person with liver disease or pathology will generally have a higher serum FAS level than a non-obese person with the same disease or pathology. Thus a high serum FAS level in a patient with inflammatory liver disease, such as viral hepatitis, may indicate the presence of steatosis or steatohepatitis in combination with the inflammatory disease.

In further embodiments, the subject's elevated FAS expression is due to increased expression by the liver, and more particularly by liver cells. Thus in some embodiments, the subject has no other source of FAS expression that contributes to a detectable or measurable elevated level of expression. A non-limiting example of an alternative source of increased FAS expression is seen in cases of cancer or other malignancy. Thus in some embodiments, the subject is one that is not afflicted by, or thought not to have, cancer or malignancy. Stated differently, the subject is one who has been diagnosed, or otherwise determined or suspected, to be cancer-free.

Thus an embodiment of the disclosure is a method of detecting elevated fatty acid synthase (FAS) in the liver of a subject. The method comprises detecting or measuring the level of fatty acid synthase (FAS) in a bodily fluid sample from a subject suspected of being, or diagnosed as, cancer-free. An elevated level of FAS indicates elevated FAS in the liver of said subject.

As disclosed herein, an elevated level of FAS in the sample indicates elevated FAS in the liver of said subject. Without being bound by theory, and offered to improve the understanding of the disclosure, the aspects and embodiments described herein are based in part on the recognition that elevated FAS expression occurs in subjects with liver conditions, like steatosis and steatohepatitis as non-limiting examples. Therefore, and in subjects with a liver condition, the detection or measurement of elevated FAS expression may be used to indicate the presence of the condition. The elevated level may thus be a marker or indicator of the condition's presence in the subject.

The increase in FAS expression seen in steatosis and steatohepatitis indicates that the source of fat accumulation results at least partially from de novo synthesis in the liver. Thus the disclosure also includes a method of detecting the liver condition known as fatty liver, or steatosis, by detecting or measuring FAS expression in a sample of one or more liver cells, a homogenate of liver cells, or a FAS containing extract of liver cells. The sample may be from a subject found or diagnosed as having an enlarged liver and/or suspected of having steatosis. Such a sample may be used in the method to diagnose or identify a subject as having, or being afflicted with, steatosis based on FAS expression. In other embodiments, an elevated FAS level in a subject with steatosis may indicate the presence of steatohepatitis not identified via liver biopsy. The methods of the disclosure allow the skilled person to distinguish between non-alcoholic steatohepatitis (NASH) and alcoholic steatohepatitis by the same criteria used in combination with liver biopsy.

The disclosure further includes a method of detecting the liver condition known as steatohepatitis by detecting or measuring FAS that has been released from the liver. In some embodiments, the release is due to hepatocellular injury which liberates FAS into a bodily fluid, such as serum. The method may comprise analysis of a sample of the bodily fluid to diagnose or identify a subject as having, or being afflicted with, steatohepatitis based upon elevated FAS level in the fluid. Thus one non-limiting embodiment includes use of a sample of serum from a subject to detect or measure elevated FAS levels which indicate the presence of steatohepatitis. Optionally, the method may be performed in combination with one or more additional assays, such as the detection of liver cancer or steatosis as described herein.

In a related aspect, the disclosure includes a method of detecting or measuring FAS expression in liver. In some embodiments, the method may comprise detecting or measuring the amount of FAS protein, or a fragment thereof, in a sample comprising liver cells, a homogenate of liver cells, or a FAS containing extract of liver cells. Optionally, the sample is all or part of a liver biopsy, such as a needle biopsy. In some embodiments, the sample is screened for the presence of cancer, such as prior to the detection of FAS levels via immunohistochemistry methods. The sample may optionally also be screened for the presence of steatosis by histological means, such as routine hematoxylin and eosin staining followed by analysis.

In other embodiments, the method may comprise detecting or measuring the amount of FAS protein, or a fragment thereof, in a sample of bodily fluid of a subject. As used herein, an FAS protein fragment is a portion of the FAS protein that is indicative of the presence or level of FAS protein. In some embodiments, the fragment is detected by a reagent that also detects FAS protein.

In some embodiments, a detected or measured level of FAS expression may be determined to be elevated by comparison to FAS expression in a normal subject free of a liver condition and so free of liver disease or pathology. In other embodiments, the comparison may be in a subject with normal physiological expression of FAS.

Where a disclosed method comprises determining the level of FAS expression, the level may be determined to be elevated relative to the level in a subject without a liver condition. This allows the subject being tested to be identified as having a liver condition, such as a liver disease or pathology. Alternatively, the level may be determined to be the same, and so not elevated, relative to the level in a subject without liver disease or pathology. This identifies the subject being tested as not having a liver condition.

Therefore, embodiments of the disclosure include a method of identifying the presence or absence of a liver condition, such as liver disease or pathology, in a subject by analysis of FAS expression. The method may comprise

detecting or measuring the level of fatty acid synthase (FAS) in a bodily fluid sample from said subject, wherein

i) a level of FAS that is elevated, relative to the level in a subject without liver disease or pathology, identifies the presence of a liver condition in said subject, and

ii) a level of FAS that is the same, relative to the level in a subject without liver disease or pathology, identifies the absence of a liver condition in said subject.

In some embodiments, the subject is suspected of being afflicted with a liver disease or pathology characterized by elevated FAS levels, such as steatohepatitis, including non-alcoholic steatohepatitis or alcoholic hepatitis. In other embodiments, the disease or pathology is not characterized by elevated FAS levels, such as liver toxicity; viral infection of the liver, or viral hepatitis; autoimmune hepatitis; cryptogenic cirrhosis; hepatic necrosis following hypoperfusion; and hepatitis resulting from other disease. In other embodiments, the subject is suspected of being afflicted with a liver disease or pathology, optionally before the onset of symptoms of cirrhosis.

In the disclosed methods, it is possible that the subject has fatty liver coincident with one or more additional conditions which elevate FAS in serum or one or more other bodily fluids. Where the elevation of FAS is from the liver, the additional condition(s) is/are a liver condition as described herein. If FAS elevation is from a non-liver source, then a skilled person may distinguish the fatty liver condition from the non-liver condition by methods known in the field, such as histological (or histopathological) examination of the liver as a non-limiting example.

In embodiments where the disease or pathology is liver toxicity, it may be due to exposure of the subject to an exogenous drug or chemical agent. In some cases, the drug or chemical agent is tetracycline or carbon tetrachloride. Alternatively, and where the disease or pathology is due to viral infection or viral hepatitis, it may be the result of a virus selected from Hepatitis A, B, C, or D. In other embodiments, the virus is cytomegalovirus or a herpesvirus, or a systemic or local viral infection.

In embodiments where the disease or pathology is autoimmune related or based, it may be due to systemic lupus erythematosis, scleroderma, CREST syndrome, or other autoimmune disease. Where the disease or pathology is hepatic necrosis following hypoperfusion, it may be severe hypotension, mechanical injury to the liver, or vascular compromise. Alternatively, the disease or pathology may be due to ulcerative colitis, Crohn's disease, sclerosing cholangitis, or cryptogenic cirrhosis.

In further embodiments, the disease or pathology may be focal or diffuse infiltration of the liver from a malignant disease. Non-limiting examples of such a malignant disease include hepatocellular carcinoma, leukiemia, lymphoma, or other primary or metastatic malignant tumors.

As described herein, the disclosed methods may be practiced by use of a reagent that binds FAS protein, or a fragment thereof. In some embodiments, the reagent is an antibody directed against FAS, such as an antibody against human fatty acid synthase (hFAS). As used herein, “antibody” refers to an immunoglobulin molecule, and fragments thereof, which are immunologically reactive with a particular antigen. In some cases, the antibody may react with FAS from human cancer cells while also reacting with liver FAS from non-transformed cells. In some cases, a monoclonal antibody, or FAS binding fragment thereof, may be used in the disclosed methods.

The term “antibodies” refers to a plurality of such molecules and is not limited to homogeneous populations of a single type of antibody. The term “antibody” also includes genetically engineered forms such as chimeric antibodies (e.g., humanized murine antibodies), heteroconjugate antibodies (e.g., bispecific antibodies), and recombinant single chain Fv fragments (scFv), and disulfide stabilized (dsFv) Fv fragments (see, for example U.S. Pat. No. 5,747,654). The term “antibody” also includes antigen binding forms of antibodies (e.g., Fab′, F(ab′)₂, Fab, Fv and rIgG. See also, Pierce Catalog and Handbook, 1994-1995 (Pierce Chemical Co., Rockford, Ill.). The term “anti-hFAS” refers to an antibody which is generated against hFAS.

An antibody immunologically reactive with FAS or hFAS as described herein can be generated by known methodologies such as immunization of an antibody producing animal with an FAS or hFAS polypeptide. Monoclonal antibodies may be obtained by various techniques familiar to those skilled in the art. Description of techniques for preparing such monoclonal antibodies may be found in, e.g., Stites, et al. (eds.) BASIC AND CLINICAL IMMUNOLOGY (4TH ED.), Lange Medical Publications, Los Altos, Calif., and references cited therein; Harlow & Lane, supra; Goding, MONOCLONAL ANTIBODIES: PRINCIPLES AND PRACTICE (2D ED.), Academic Press, New York, N.Y. (1986); Kohler & Milstein, Nature 256:495-497 (1975); and particularly (Chowdhury, P. S., et al. Mol. Immunol. 34:9 (1997)), which discusses one non-limiting method of generating monoclonal antibodies.

Methods to prepare monoclonal antibodies include the immunization of an animal with a nucleic acid sequence that encodes the desired immnunogen, in this case, an FAS polypeptide. This technique has at least two advantages over protein-based immunization: avoidance of the need for protein purification; and increased likelihood of proper post-translational modification of the immunogen.

In some embodiments, the antibody may be referred to as being “specific for” or “specifically immunoreactive with” an FAS protein or a fragment thereof. These terms refer to the ability of the antibody to react in a binding reaction to an FAS, such as human FAS, or a fragment thereof. The reaction can be determinative of the presence or amount of FAS in the presence of other proteins, cells, or materials. Under assay conditions as desired by the skilled practitioner, including the non-limiting conditions disclosed herein, the antibody binds preferentially to FAS and does not bind in a significant or detectable manner to other factors in a sample. Non-limiting embodiments of the disclosure utilize conditions wherein the antibody, or an alternative form thereof, selectively binds to produce a signal which is at least two times, three times, four times, five times, six times, seven times, eight times, nine times, or at least 10 times to 100 times, background signal or noise. Background signal or noise may include low level cross reactivity with other proteins or biological materials.

Alternative forms of the FAS specific antibodies of the disclosure can be readily produced by methods known to the skilled person. The ability to produce antigen binding fragments of antibodies is well known and may be utilized to produce bivalent F(ab′)₂ and monovalent Fab fragments for use as disclosed herein. As used herein, “Fab” refers to double chain binding fragments of antibodies comprising at least functionally complete light and heavy chain variable domains. Additionally, methods for the production of hybrid, chimeric, altered, recombinant (including single chain), or humanized forms of antibodies are also known to the skilled person. These antibody forms may be considered derivatives of the antibodies and monoclonal antibodies disclosed herein.

In some embodiments, the alternative form is a “single chain Fv” or “scFv”, which refer to an antibody in which a heavy chain and a light chain of a traditional two chain antibody have been joined to form one chain with a single binding site. Typically, a linker peptide is placed between the two chains to allow for proper folding and positioning of the variable region to create the active binding site. The term “linker peptide” refers to a polypeptide chain within an antibody binding fragment (e.g., Fv fragment) which serves to indirectly attach the variable heavy chain to the variable light chain.

More generally, a “linker” is a molecule used to join the antibody to another molecule. The linker is capable of forming covalent bonds to both the antibody and to the other molecule. Suitable linkers are well known to the skilled person and include, but are not limited to, straight or branched chain carbon linkers, hetero cyclic carbon linkers, or peptide linkers. Where the antibody and another molecule are polypeptides, the linkers may be joined to the constituent amino acids through their side groups (e.g., through a disulfide linkage to cysteine). Alternatively, the linkers will be joined to the alpha carbon amino and carboxyl groups of the terminal amino acids.

Additional derivative forms include antibodies of the disclosure, and alternative forms thereof, that have been conjugated to other chemical moieties. Non-limiting examples include a labeled antibody or an alternative form thereof. The term “label”, “detectably labeled” or “labeled with a detectable marker” refer to an antibody composition capable of being detected (directly or indirectly) to indicate the presence of the “labeled” molecule. The detection may be made quantitatively or qualitatively. Thus a label produces a detectable signal indicative of the presence of the labeled molecule, and a labeled antibody of the disclosure may be detected by virtue of the label. Suitable labels include one member of a binding pair (such as biotin in a biotin-avidin or biotin-strepavidin binding pair), a radioisotope, a chromophore, an enzyme (e.g., horse radish peroxidase, alkaline phosphatase and others commonly used in an ELISA), a substrate, a dye, a fluorescent molecule (e.g., fluorescein isothiocyanate, Texas red, rhodamine, green fluorescent protein, and the like), a chemiluminescent moiety, a magnetic particle or bead, a bioluminescent moiety, a calorimetric label such as colloidal gold, and the like. As such, a label is any composition detectable, directly or indirectly, by spectroscopic, photochemical, biochemical, immunochemical, electrical, optical or chemical means. In some embodiments, the label produces a visible signal that can be detected by visual inspection, such as by the unaided eye.

The means to detect such labels are well known to the skilled person. For example, radiolabels may be detected using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and calorimetric labels are detected by simply visualizing the colored label.

The antibodies of the disclosure and alternative forms thereof may also be conjugated by known methods and means to a solid phase support such as, but not limited to, glass, plastic, a synthetic membrane. Other non-limiting examples include beads, particles, dipsticks, fibers, filters, Petri dishes, ELISA (enzyme-linked immunosorbent assay) plates, microtiter plates, silane or silicate supports such as glass slides, and dishes, wells or containers, as well as the sides thereof. Such immobilized forms of the antibodies may be used in the detection methods disclosed herein.

The antibodies of the disclosure and alternative forms thereof may also be formulated into compositions. The compositions may further comprise one or more other reagent for the detection of FAS or a fragment thereof. Non-limiting examples include complexes of the antibody bound to its cognate FAS and combinations of the antibody with other reagents for use in antibody based detection methods. Other examples include mixtures with other FAS binding antibodies or detection agents. Combinations of the antibodies, and alternative forms thereof, with other detection agents may also be part of articles of manufacture, such as testing devices, used to detect FAS.

The methods used to detect or measure FAS, or a fragment thereof, are not limited by design. Non-limiting examples include methods utilizing the antibodies of the disclosure, and alternative forms thereof as described herein, and based upon the principles of Western blotting or other immunoblotting, ELISA, lateral flow devices, sandwich assays, visual observation by microscopy, competitive and non-competitive immunoassays, immunoenzymetric assays, immunofluorescence, immunomagnetic selection, and flow cytometry (including detection by polychromatic flow cytometry). Additional immunoassay formats are described by Harlow and Lane (1988) Antibodies, A Laboratory Manual, Cold Spring Harbor Publications, New York. The methods of the disclosure are used to qualitatively or quantitatively detect or measure the presence or absence of FAS in a sample or “test sample” as described herein.

As used herein, a “sample” or “test sample” refers to a sample isolated from an individual suspected of having elevated FAS or a condition which includes elevated FAS as a detectable sign. Alternatively, the terms refer to samples known to contain FAS, such as human FAS, for use as a control in the disclosed detection methods or for use in the disclosed detection methods to confirm the presence of, or quantify the amount of, FAS. The sample may be collected by any appropriate means, including biopsy and needle biopsy in the case of a cell containing sample, and blood collection in the case of a serum containing sample. A sample of the disclosure may also be a homogenate or extract of liver cells as non-limiting examples. A sample may also be a dilution of a serum, such as dilution with a sample diluent before being assayed. The diluent may be any suitable solvent as desired by the skilled person.

In one embodiment, a method of the disclosure is based on the use of a capture reagent which binds FAS, or a fragment thereof, to form a complex therewith. The capture reagent may be the monoclonal antibody, or alternative forms thereof, as described herein. Alternatively, the reagent may be another antibody which binds FAS, including, but not limited to, polyclonal or recombinant antibodies that bind a plurality of FAS and other cellular components. The capture reagent may be immobilized on a solid phase support, optionally prior to contact with a sample of the disclosure, as described herein for antibodies of the disclosure. Of course capture agents that bind a complex of FAS protein and an FAS specific antibody, rather than the antibody alone, may also be used in the practice of the disclosure.

Whether used with a capture reagent or not, the disclosure also includes a detection agent that binds FAS protein, or a fragment thereof, to directly or indirectly indicated its presence or amount. The detection agent may be an antibody of the disclosure, or an alternative form thereof, which binds FAS protein or a fragment thereof. Upon binding, the detection agent forms a bound complex with its binding partner. The detection agent may be detectably labeled such that the presence or amount of the cognate binding partner, and thus FAS protein or a fragment thereof, is signaled by the label after binding of the detection agent. Alternatively, the detection agent is itself bound by a detectably labeled secondary agent.

When used in combination with a capture reagent, a sandwich complex comprising the reagent, an FAS protein or fragment thereof, and the detection agent is formed. This sandwich complex may be preceded by formation of a complex comprising the capture reagent and an FAS protein or fragment thereof, which complex is exposed to the detection agent to form the sandwich complex. Alternatively, the sandwich complex may be preceded by formation of a complex comprising the detection reagent and an FAS protein or fragment thereof, which complex is subsequently exposed to the capture reagent to form the sandwich complex. The specificity of the sandwich complex, as well as other formats, can be introduced by either the capture reagent, the detection reagent, or both.

Embodiments of the disclosure include use of the following combinations of capture reagent and detector reagent:

polyclonal antibodies that bind FAS protein or fragment thereof, and monoclonal antibody that binds FAS protein or fragment thereof;

polyclonal antibodies that bind a complex comprising FAS protein or fragment thereof, and monoclonal antibody that binds FAS protein or fragment thereof;

monoclonal antibody that binds FAS protein or fragment thereof, and

polyclonal antibodies that bind FAS protein or fragment thereof;

monoclonal antibody that binds FAS protein or fragment thereof, and polyclonal antibodies that bind a complex comprising FAS protein or fragment thereof; and

monoclonal antibody that binds FAS protein or fragment thereof, and monoclonal antibody that binds FAS protein or fragment thereof.

The methods of the disclosure also include competitive binding assays as embodiments. These comprise the use of a labeled form of FAS protein or fragment thereof that competes for binding to a detection agent and/or capture reagent as described herein and analogous to competitive assay methods known in the art. The methods provided by this disclosure may also be automated in whole or in part.

The materials for use in the methods of the disclosure are ideally suited for preparation of kits produced in accordance with well known procedures. The disclosure includes kits comprising agents for the detection and/or quantitation of FAS protein or fragment thereof, in a sample as described herein. Such kits optionally comprising the agents and/or reagents with an identifying description or label or instructions relating to the use of the kits, or the suitability of the kits, in a method of the disclosure. Such a kit may comprise containers, each with one or more of the various agents and/or reagents (optionally in concentrated form) utilized in the methods, including, for example, detection agents and/or pre-immobilized forms of capture reagents. A set of instructions or reagent identifiers will also typically be included. Other exemplary kits contain a device or solid phase supports, such as, but not limited to a lateral flow device, a test strip, beads, a membrane, or coated surfaces of a container, dish or well, for the practice of the disclosed method(s).

The kits may also optionally include a control sample, such as a known sample of immunoreactive FAS protein or fragment thereof. A control can be present in known quantities for dilution with the sample diluent used to dilute a sample and used as an external control or added to an actual sample and used as an internal control, optionally for use to determine the sensitivity of the assay in the context of the sample type being tested. The kits can comprise materials for a single assay or for multiple assays.

Therefore, embodiments of the disclosure include a method comprising the forming and then detecting or measuring of a complex between a FAS binding antibody, or FAS binding fragment thereof, and FAS protein or fragment thereof. In some embodiments, the complex may be formed by contacting the FAS binding antibody, or FAS binding fragment thereof, with a sample that may contain FAS protein or fragment thereof. The contacting occurs under conditions which allow the formation of the complex if FAS is present in the sample. As disclosed herein, the sample may be a liver cell containing, or derived, sample or a sample of bodily fluid. In further embodiments, the sample may be a positive control which contains FAS.

In some cases, the detecting or measuring is conducted by use of an enzyme-linked immunosorbent assay (ELISA) or radioimmuno assay. Alternatively, the detecting or measuring is by use of a lateral flow device wherein a complex as described above may be isolated on a solid phase component of the lateral flow device.

In another aspect, the disclosure includes a method of detecting fat accumulation in the liver of a subject. In some embodiments, the method may comprise detecting or measuring FAS expression in a sample comprising one or more liver cells, a homogenate of liver cells, or a FAS containing extract of liver cells from a subject. The level of FAS expression may be determined to be elevated as described herein. An elevated level may be used to indicate accumulation of de novo synthesized fat in the liver of the subject.

In further embodiments, this method may further comprising detecting liver inflammation and/or hepatocellular injury. The absence of inflammation or injury indicates the presence of steatosis while the presence of inflammation or injury indicates the presence of steatohepatitis.

In additional embodiments, an elevated level of FAS expression that indicates the presence of fat accumulation may also be used to indicate that greater than about 5% of the total liver weight, or more than about 30% of liver cells in a liver lobule, are with fat deposit. In further embodiments, the level may indicate that greater than about 6%, about 7%, about 8%, about 9%, about 106% or more of the total liver weight, or more than about 32%, about 34%, about 36%, about 38%, about 40% or more of liver cells in a liver lobule, are with fat deposit.

The disclosed methods may comprise additional acts. In some embodiments, the additional act is communicating the level of FAS expression to said subject. In other embodiments, the additional act is requesting or receiving payment for detecting or measuring FAS expression.

Having now generally set forth the disclosure, the same will be more readily understood through reference to the following examples which are provided by way of illustration, and are not intended to be limiting of the present disclosure, unless specified.

### EXAMPLES

### Example 1

To examine the relationships among steatosis, steatohepatitis and FAS expression, FAS expression was studied in needle biopsies from 38 obese subjects with immunohistochemistry with a monoclonal anti-human FAS antibody. Steatosis, steatohepatitis, and FAS expression were graded semi-quantitatively from 0-4+ with increments of 0.5. Analysis of the data revealed that steatosis and FAS expression were best grouped into three categories: 0-0.5+ which represented negative or scant expression, 1.0-2.5+ which indicated a mild to moderate expression, and 3.0-4.0 indicating strong expression. FIG. 2 shows the results of this study.

FIG. 2, Panel A compares the association between steatohepatitis and fatty liver. As expected, patients without fatty liver (0-0.5+) have little steatohepatitis, while those with marked steatosis (3.0-4.0+) have the highest level of steatohepatitis p<0.0001 (1-way ANOVA analysis, Prism 4.00 for Windows, GraphPad Software, San Diego Calif. USA, www.graphpad.com). These findings are consistent with the known association between fatty liver and steatohepatitis (2).

FIG. 2, Panel B compares the relationship between fatty liver (steatosis) and FAS expression. There is an association between increasing steatosis and FAS expression (p=0.011). Without being bound by theory, and offered to improve the understanding of the disclosure, these results suggest that some or most of the fat accumulated in the liver may be the product of de novo fatty acid synthesis within the liver rather than representing mobilization and transport of fatty acids from adipose tissue to the liver.

FIG. 2, Panel C compares FAS expression to active steatohepatitis. There is a strong relationship between increased FAS expression and the presence of steatohepatitis (p=0.0003). Thus, similar to fatty liver, FAS expression is also highly associated with the presence of steatohepatitis. Again without being bound by theory, and offered to improve the understanding of the disclosure, these results suggest that there is a relationship between steatohepatitis and elevated blood and serum levels of FAS.

### Example 2

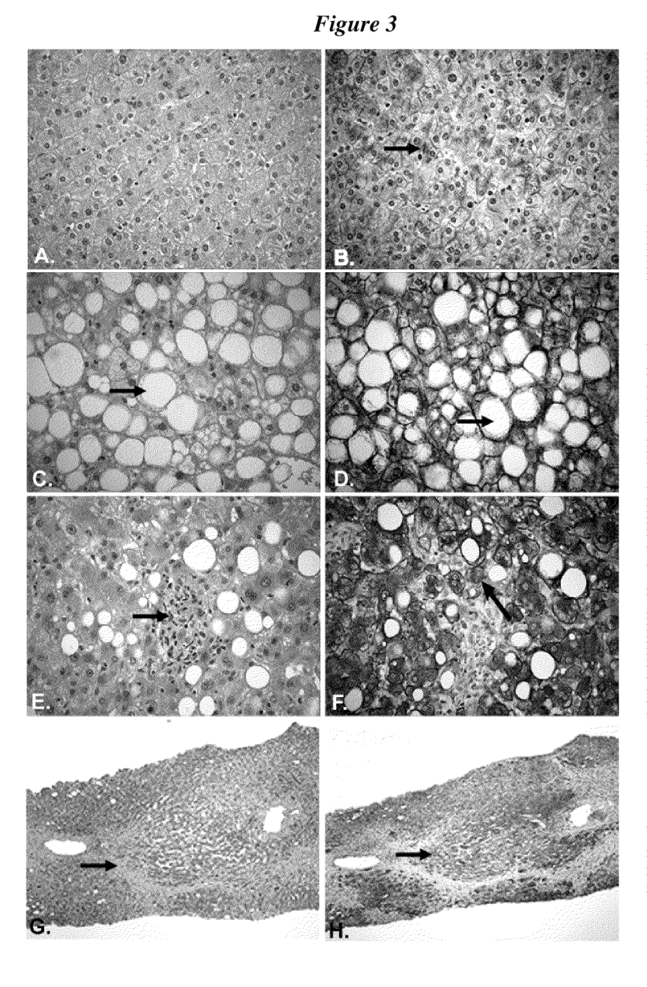

FIG. 3 is a series of photomicrographs illustrating the association of FAS expression with steatosis, steatohepatitis, and cirrhosis. FIG. 3A is a hematoxylin and eosin stained section of formalin-fixed paraffin-embedded liver of histologically unremarkable liver without evidence of steatosis or steatohepatitis. In 3B an approximate serial section of the normal liver biopsy was stained with an anti-human FAS mouse monoclonal antibody. There is slight reactivity for FAS in this liver as noted by the brick colored staining which corresponds to FAS expression (arrow). In contrast, FIG. 3C is a hematoxylin and eosin stained section of liver with severe steatosis. The large round spaces which represent triglyeride droplets which engorge the liver cell (arrow) should be noted. This is an example of macrovesicular steatosis. In addition, there are a few cells with small vacuoles which render a ‘soap bubble’ appearance to the cytoplasm indicative of a component of microvesicular steatosis.

FIG. 3D illustrates immunohistochemistry for FAS in the same biopsy. The intense cytoplasmic staining for FAS in cells with abundant triglyceride (arrow) indicating up-regulation of FAS expression compared to the normal liver in 3B should be noted. Steatohepatitis is illustrated in FIG. 3E. The focus of inflammation among the hepatocytes in an area of predominantly macrovesicular steatosis (arrow) should be noted.

Immuno-histochemical staining for FAS in 3F shows intense cytoplasmic staining for FAS in all the hepatocytes, even those caught up in the inflammation (arrow). Patients with steatohepatitis may go on to develop cirrhosis. FIG. 3G is a hematoxylin and eosin stained liver biopsy from a patient with advanced cirrhosis which developed in the setting of steatohepatitis. The dense fibrous scarring that creates the nodular appearance of cirrhosis (arrow) should be noted. High levels of FAS expression remain in this cirrhotic liver as shown in FIG. 3H (arrow).

In summary, high levels of FAS expression appears associated with steatosis and steatohepatitis on through to the development of established cirrhosis in patients with NASH.

### Example 3

To determine the relationship between steatosis or steatohepatitis have detectable circulating FAS, FAS was measured in discarded pre-surgical sera from 16 patients undergoing bariatric surgery for morbid obesity. In addition, surgical liver biopsies were also performed on 12 of these patients to assess the presence of steatosis or steatohepatitis. Serum FAS levels were measured using a monoclonal anti-human FAS sandwich ELISA assay. Compared to 20 normal subjects, the 16 obese patients had higher levels of serum FAS as shown in FIG. 4A (3.5±1.17 ng/ml, obese; 0.17±0.07 ng/ml, normal; p=0.003, two-tailed unpaired t-test, GraphPad Prism version 4.00 for Windows, GraphPad Software, San Diego Calif. USA, www.graphpad.com).

For the twelve patients, from whom liver tissue was available from surgery, steatohepatitis and steatosis were graded using a 0-4+ semiquantitative scale based on the degree of fatty change, inflammation, and fibrosis. Increased serum FAS levels were associated with steatohepatitis, not steatosis (p=0.03, one-tailed t-test, Prism 4.0, Graph Pad Software). FIG. 4B is a graphical depiction of elevated serum FAS levels in patients with steatohepatitis compared to those without steatohepatitis.

On the 17 patients with serum FAS measurements, a battery of liver function tests was performed. The tests included ALT, AST, ALP, total bilirubin, total protein, and albumin. Using a Pearsons correlation test, these analytes showed no significant correlation with serum FAS values. This suggests that serum FAS levels are likely independent of this battery of routine liver function studies.

All references cited herein, including patents, patent applications, and publications, are hereby incorporated by reference in their entireties, whether previously specifically incorporated or not.

Having now fully provided the instant disclosure, it will be appreciated by those skilled in the art that the same can be performed within a wide range of equivalent parameters, concentrations, and conditions without departing from the spirit and scope of the disclosure and without undue experimentation.

While the disclosure has been described in connection with specific embodiments thereof, it will be understood that it is capable of further modifications. This application is intended to cover any variations, uses, or adaptations of the disclosure following, in general, the disclosed principles and including such departures from the disclosure as come within known or customary practice within the art to which the disclosure pertains and as may be applied to the essential features hereinbefore set forth.

### BIBLIOGRAPHY

1. Clark, J. M. The epidemiology of nonalcoholic Fatty liver disease in adults. J Clin Gastroenterol, 40 Suppl 1: S5-S10, 2006.
2. Brunt, E. M. Nonalcoholic steatohepatitis: definition and pathology. Semin Liver Dis, 21: 3-16, 2001.
3. Younossi, Z. M., Gramlich, T., Liu, Y. C., Matteoni, C., Petrelli, M., Goldblum, J., Rybicki, L., and McCullough, A. J. Nonalcoholic fatty liver disease: assessment of variability in pathologic interpretations. Mod Pathol, 11: 560-565, 1998.
4. Ratziu, V., Charlotte, F., and Heurtier, A. High sampling variability of percutaneous liver biopsy in non-alcoholic fatty livers. Hepatology, 40: 237A, 2004.
5. Henry, J. B. Clinical diagnosis and management by laboratory methods, 20 edition, p. 268-269. Philadelphia: W.B. Saunders Company, 2001.
6. Kunde, S. S., Lazenby, A. J., Clements, R. H., and Abrams, G. A. Spectrum of NAFLD and diagnostic implications of the proposed new normal range for serum ALT in obese women. Hepatology, 42: 650-656, 2005.
7. Amarapurkar, D. N. and Patel, N. D. Clinical spectrum and natural history of non-alcoholic steatohepatitis with normal alanine aminotransferase values. Trop Gastroenterol, 25: 130-134, 2004.
8. Fassio, E., Alvarez, E., Dominguez, N., Landeira, G., and Longo, C. Natural history of nonalcoholic steatohepatitis: a longitudinal study of repeat liver biopsies. Hepatology, 40: 820-826, 2004.
9. Garcia-Monzon, C., Martin-Perez, E., Iacono, O. L., Fernandez-Bermejo, M., Majano, P. L., Apolinario, A., Larranaga, E., and Moreno-Otero, R. Characterization of pathogenic and prognostic factors of nonalcoholic steatohepatitis associated with obesity. J Hepatol, 33: 716-724, 2000.
10. Mofrad, P., Contos, M. J., Haque, M., Sargeant, C., Fisher, R. A., Luketic, V. A., Sterling, R. K., Shiffman, M. L., Stravitz, R. T., and Sanyal, A. J. Clinical and histologic spectrum of nonalcoholic fatty liver disease associated with normal ALT values. Hepatology, 37: 1286-1292, 2003.
11. Torezan-Filho, M. A., Alves, V. A., Neto, C. A., Fernandes, H. S., and Strauss, E. Clinical significance of elevated alanine aminotransferase in blood donors: a follow-up study. Liver Int, 24: 575-581, 2004.
12. Kuhajda, F. P. Fatty-acid synthase and human cancer: new perspectives on its role in tumor biology. Nutrition, 16: 202-208, 2000.
13. Pizer, E. S., Jackisch, C., Wood, F. D., Pasternack, G. R., Davidson, N. E., and Kuhajda, F. P. Inhibition of fatty acid synthesis induces programmed cell death in human breast cancer cells. Cancer Res, 56: 2745-2747, 1996.
14. Pizer, E. S., Thupari, J., Han, W. F., Pinn, M. L., Chrest, F. J., Frehywot, G. L., Townsend, C. A., and Kuhajda, F. P. Malonyl-coenzyme-A is a potential mediator of cytotoxicity induced by fatty-acid synthase inhibition in human breast cancer cells and xenografts. Cancer Res, 60: 213-218, 2000.
15. Pizer, E. S., Pflug, B. R., Bova, G. S., Han, W. F., Udan, M. S., and Nelson, J. B. Increased fatty acid synthase as a therapeutic target in androgen-independent prostate cancer progression. Prostate, 47: 102-110, 2001.
16. Wang, Y., Kuhajda, F. P., Li, J. N., Pizer, E. S., Han, W. F., Sokoll, L. J., and Chan, D. W. Fatty acid synthase (FAS) expression in human breast cancer cell culture supernatants and in breast cancer patients. Cancer Lett, 167: 99-104, 2001.
17. Wang, Y. Y., Kuhajda, F. P., Cheng, P., Chee, W. Y., Li, T., Helzlsouer, K. J., Sokoll, L. J., and Chan, D. W. A new model ELISA, based on two monoclonal antibodies, for quantification of fatty acid synthase. J Immunoassay Immunochem, 23: 279-292, 2002.
18. Wang, Y. Y., Kuhajda, F. P., Li, J., Finch, T. T., Cheng, P., Koh, C., Li, T., Sokoll, L. J., and Chan, D. W. Fatty acid synthase as a tumor marker: its extracellular expression in human breast cancer. J Exp Ther Oncol, 4: 101-110, 2004.
19. Wang, Y., Kuhajda, F. P., Sokoll, L. J., and Chan, D. W. Two-site ELISA for the quantitative determination of fatty acid synthase. Clin Chim Acta, 304: 107-115, 2001.

What is claimed is:

1. A method of detecting elevated fatty acid synthase (FAS) in the liver of a subject, said method comprising
detecting or measuring the level of fatty acid synthase (FAS) in a bodily fluid sample from a subject suspected of being, or diagnosed as, cancer-free,
wherein an elevated level of FAS indicates elevated FAS in the liver of said subject.

2. The method of claim 1 further comprising identifying the presence or absence of liver disease in said subject, wherein
i) a level of FAS that is elevated, relative to the level in a subject without liver disease, identifies the presence of liver disease in said subject, and
ii) a level of FAS that is the same, relative to the level in a subject without liver disease, identifies the absence of liver disease in said subject.

3. The method of claim 2 wherein said liver disease is characterized by an elevated level of FAS is steatohepatitis, such as non-alcoholic steatohepatitis or alcoholic hepatitis; or
not characterized by an elevated level of FAS, such as liver toxicity; viral infection of the liver, or viral hepatitis; autoimmune hepatitis; cryptogenic cirrhosis; hepatic necrosis following hypoperfusion; and hepatitis resulting from other disease.

4. The method of claim 3 wherein
said liver toxicity is due to exposure of the subject to an exogenous drug or chemical agent, such as tetracycline or carbon tetrachloride;
said viral infection or viral hepatitis is selected from Hepatitis A, B, C, or D, or is due to infection by cytomegalovirus or herpesvirus;
said autoimmune hepatitis is due to systemic lupus erythematosis, scleroderma, or CREST syndrome in said subject;
said hepatic necrosis following hypoperfusion is selected from severe hypotension, mechanical injury to the liver, or vascular compromise; or
said hepatitis resulting from other disease is selected from ulcerative colitis, Crohn's disease, or sclerosing cholangitis.

5. The method of claim 1 wherein said subject is human.

6. The method of claim 1 wherein said bodily fluid is blood or serum.

7. The method of claim 1 wherein said detecting or measuring comprises forming and detecting or measuring a complex between a FAS binding antibody, or antibody fragment, and FAS, if present, in said sample.

8. The method of claim 7, wherein said detecting or measuring is by ELISA or a lateral flow test strip.

9. The method of claim 1 further comprising communicating the level of FAS to said subject.

10. The method of claim 2 wherein said subject is suspected of being afflicted with a liver disease, optionally before the onset of symptoms of cirrhosis.

11. A method of detecting elevated fatty acid synthase (FAS) in the liver of a subject, said method comprising
detecting or measuring the level of fatty acid synthase (FAS) expression in a sample comprising liver cells from a subject,
wherein an elevated level of FAS expression indicates elevated FAS in the liver of said subject.

12. The method of claim 11 further comprising identifying the presence or absence of liver disease in said subject, wherein
i) a level of FAS that is elevated, relative to the level in a subject without liver disease, identifies the presence of liver disease in said subject, and
ii) a level of FAS that is the same, relative to the level in a subject without liver disease, identifies the absence of liver disease in said subject.

13. The method of claim 12 wherein said liver disease is selected from steatohepatitis (non-alcoholic steatohepatitis or alcoholic hepatitis); alcoholic hepatitis; liver toxicity; viral infection of the liver, or viral hepatitis; autoimmune hepatitis; cryptogenic cirrhosis; hepatic necrosis following hypoperfusion; and hepatitis resulting from other disease.

14. The method of claim 13 wherein
said liver toxicity is due to exposure of the subject to an exogenous drug or chemical agent, such as tetracycline or carbon tetrachloride;
said viral infection or viral hepatitis is selected from Hepatitis A, B, C, or D, or is due to infection by cytomegalovirus or herpesvirus;
said autoimmune hepatitis is due to systemic lupus erythematosis, scleroderma, or CREST syndrome in said subject;
said hepatic necrosis following hypoperfusion is selected from severe hypotension, mechanical injury to the liver, or vascular compromise; or
said hepatitis resulting from other disease is selected from ulcerative colitis, Crohn's disease, or sclerosing cholangitis.

15. The method of claim 11 wherein said subject is human; or
said bodily fluid is blood or serum; or
further comprising communicating the level of FAS to said subject.

16. The method of claim 11 wherein said detecting or measuring comprises forming and detecting or measuring a complex between a FAS binding antibody, or antibody fragment, and FAS, if present, in said sample.

17. The method of claim 16, wherein said detecting or measuring is by ELISA or a lateral flow test strip.

18. The method of claim 12 wherein said subject is suspected of being afflicted with a liver disease, optionally before the onset of symptoms of cirrhosis.

19. A method of detecting fat accumulation in the liver of a subject, said method comprising
detecting or measuring the level of fatty acid synthase (FAS) expression in a sample comprising liver cells from a subject,
wherein an elevated level of FAS indicates accumulation of fat synthesized de novo in the liver of said subject.

20. The method of claim 20 further comprising detection of liver inflammation, wherein the absence of inflammation indicates the presence of steatosis and the presence of inflammation indicates the presence of steatohepatitis; or
wherein said level of FAS expression indicates that greater than about 5% of the total liver weight, or more than about 30% of liver cells in a liver lobule, are with fat deposit.
